(19) United States
(12) Patent Application Publication
(10) Pub. No.: US 2007/0038067 A1
(43) Pub. Date: Feb. 15, 2007

(54) Living body inspection apparatus

(75) Inventors: Akihiko Kandori, Tokyo (JP); Tsuyoshi Miyashita, Fuchu (JP); Kuniomi Ogata, Kokubunji (JP); Atsushi Maki, Fuchu (JP)

Correspondence Address: Stanley P. Fisher;Reed Smith LLP, Suite 1400, 3110 Fairview Park Drive, Falls Church, VA, 22042-4503, US

(73) Assignee: Hitachi, Ltd.

(21) Appl. No.: 11/480,518

(22) Filed: Jul. 5, 2006

(30) Foreign Application Priority Data
Jul. 26, 2005 (JP) 2005-216534

(51) Int. Cl. A61B 5/05

(52) U.S. Cl. 600/409

(57) ABSTRACT

Living body inspection apparatus which concurrently carries out proper measurement of movement of a living body and measurement of a magnetic field emanating from the living body. It includes a movement sensor which includes magnetic field generating means for generating a magnetic field and magnetic field receiving means attached to a living body for receiving the magnetic field and measures movement of the living body based on the magnetic field emitted from the magnetic field generating means and received by the magnetic field receiving means. It also includes: a SQUID magnetometer which receives a biomagnetic field emanating from the living body; analyzing means which analyzes measurement data obtained by the movement sensor and the SQUID magnetometer; and display means which outputs a result of analysis made by the analyzing means. The magnetic field generating means is fixed in a prescribed position relative to the SQUID magnetometer.

### CLAIM OF PRIORITY

The present application claims priority from Japanese application JP 2005-216534 filed on Jul. 26, 2005, the content of which is hereby incorporated by reference into this application.

### FIELD OF THE INVENTION

The present invention relates to living body inspection apparatus which measures the motor function of a living body and more particularly to living body inspection apparatus which concurrently carries out measurement of the movement of a living body and measurement of a magnetic field emanating from the living body.

### BACKGROUND OF THE INVENTION

Parkinson's disease is an incurable disease which develops a lesion in the substantia nigra or striate body within the brain which controls a movement, and causes ataxia of involuntary movement such as walking. Also, since it is a progressive disease, it is said that the patient becomes bedridden if the disease is untreated for about ten years. Thus, early diagnosis and treatment are needed.

However, Parkinson's disease has no pathognomonic signs which can be detected by a blood test or existing diagnostic imaging methods such as CT and MRI though it is a brain disease, and at the moment, the severity (progression stage) of the disease state is determined according to specific clinical manifestations (tremor, rigidity, akinesia, postural instability, etc.) and the patient's complaint. In the above diagnostic method, it is difficult to quantitatively evaluate the severity of the disease state, and sufficient information for appropriate medications cannot be obtained.

Conventionally, biomagnetic field equipment which uses a SQUID (Superconducting Quantum Interference Device) magnetometer has been used to measure a weak biomagnetic field (measured magnetic field is called a cardiac magnetic field or brain magnetic field) generated by a flow of ions which is caused by myocardial activity in a living body (muscular activity in general) or neuron activity in the brain.

Various biological information can be obtained by analysis of magnetic fields measured by biomagnetic field measuring equipment. For example, a brain magnetometer (magnetoencephalograph) can be used to measure spontaneous brain magnetic fields generated spontaneously in a subject, evoked brain magnetic fields evoked by electrical or mechanical stimulation given to a subject, movement-related brain magnetic fields generated by a subject's movement, and so on.

Although event-related brain magnetic fields (including the abovementioned evoked and movement-related magnetic fields) except spontaneous brain magnetic fields are usually weaker than spontaneous brain magnetic fields, the S/N (Signal/Noise) ratio can be improved by giving the subject a stimulus or task repeatedly and averaging repeated brain magnetic field data thus generated. The use of such a brain magnetometer is expected to contribute to progress in diagnosis and studies of brain diseases such as Parkinson's disease.

With this background, biomagnetic field measuring equipment for diagnosis of Parkinson's disease in which a simple device for button tapping movements is provided and brain magnetic fields generated by tapping movements are measured over time has been disclosed in the following article: Yoshino K., Takagi K., Nomura T., Sati S., and Tonoike M. MEG responses during rhythmic finger tapping in humans to phasic stimulation and their interpretation based on neural mechanisms. “Biological Cybernetics”, 86, pp. 483-496. 2002. This biomagnetic field measuring equipment evaluates finger movements as digital on/off information and measures brain magnetic fields generated by finger movements.

### SUMMARY OF THE INVENTION

However, according to the equipment disclosed in the above article, no information with respect to finger movements other than digital on/off information can be acquired, thereby making it impossible to properly determine the poorness of finger movements which is a symptom specific to Parkinson's disease. Therefore, even if button tapping movements are not uniform (for example, the opening distance between fingers differs, the tapping speed differs, a tremor occurs during tapping, or the like), averaging operation is performed on brain magnetic field data corresponding to all movements.

Therefore, an object of the present invention is to provide equipment which concurrently carries out proper measurement of the movement of a living body and measurement of a magnetic field emanating from the living body.

In order to achieve the above object, according to one aspect of the present invention, living body inspection apparatus includes a movement sensor which has magnetic field generating means for generating a magnetic field and magnetic field receiving means attached to a living body for receiving the magnetic field and measures movement of the living body based on the magnetic field emitted from the magnetic field generating means and received by the magnetic field receiving means. It also includes a SQUID magnetometer which receives a biomagnetic field emanating from the living body; analyzing means which analyzes measurement data obtained by the movement sensor and the SQUID magnetometer; and display means which outputs a result of analysis made by the analyzing means. Here, the magnetic field generating means is fixed in a prescribed position relative to the SQUID magnetometer.

This constitution makes it possible to measure the movement of a subject properly and perform averaging operation based on brain magnetic field data corresponding to uniform movement. Consequently, the relation between the brain and movement of a patient with a brain disorder such as Parkinson's disease can be evaluated properly.

Other aspects of the invention will be apparent from the following description in this specification.

According to the present invention, proper measurement of the movement of a living body and measurement of a magnetic field emanating from the living body can be carried out concurrently.

### BRIEF DESCRIPTION OF THE DRAWINGS

FIG. 1 is a perspective external view of living body inspection apparatus according to a first embodiment of the invention;

FIG. 2 is a block diagram showing the general structure of the living body inspection apparatus according to the first embodiment;

FIG. 3 is a perspective view of an array of SQUID magnetometers;

FIG. 4 is a perspective view of the structure of a SQUID magnetometer;

FIGS. 5A and 5B are top views illustrating the positional relationship between a subject's head and SQUID magnetometers where FIG. 5A shows a left temporal measuring zone and FIG. 5B shows a right temporal measuring zone;

FIGS. 6A, 6B, and 6C are perspective views of different fixing means where FIG. 6A shows the use of an adhesive agent, FIG. 6B shows the use of a flat plate, and FIG. 6C shows a transmitter coil embedded in a bed;

FIG. 7 is a block diagram showing the structure of a movement sensor controller;

FIGS. 8A and 8B illustrate the process in which averaging means in the first embodiment performs averaging operation on brain magnetic data and generates brain magnetic waveforms where FIG. 8A shows a distance waveform and FIG. 8B concerns brain magnetic data;

FIGS. 9A, 9B, and 9C show data measured by the living body inspection apparatus according to the first embodiment where FIG. 9A shows a distance waveform, FIG. 9B shows superimposed brain magnetic waveforms, and FIG. 9C is a current arrow map;

FIG. 10 is a perspective external view of living body inspection apparatus according to a second embodiment of the invention;

FIGS. 11A and 11B illustrate the process in which averaging means in the second embodiment performs averaging operation on brain magnetic data to generate a brain magnetic waveform where FIG. 11A concerns a synchronizing signal and FIG. 11B shows a distance waveform;

FIG. 12 illustrates a case that measurement is made using an electrocardiograph along with the living body inspection apparatus; and

FIG. 13 illustrates a variation of a movement sensor.

### DETAILED DESCRIPTION OF THE PREFERRED EMBODIMENTS

Next, preferred embodiments of the present invention will be described in detail with reference to the accompanying drawings.

### First Embodiment

The first embodiment measures the motor function of a subject by letting the subject do tapping quickly. Specifically, the subject is instructed to do tapping, or putting together the forefinger and the thumb as quickly as possible, and the finger movement and the magnetic field emanating from the brain during tapping are measured.

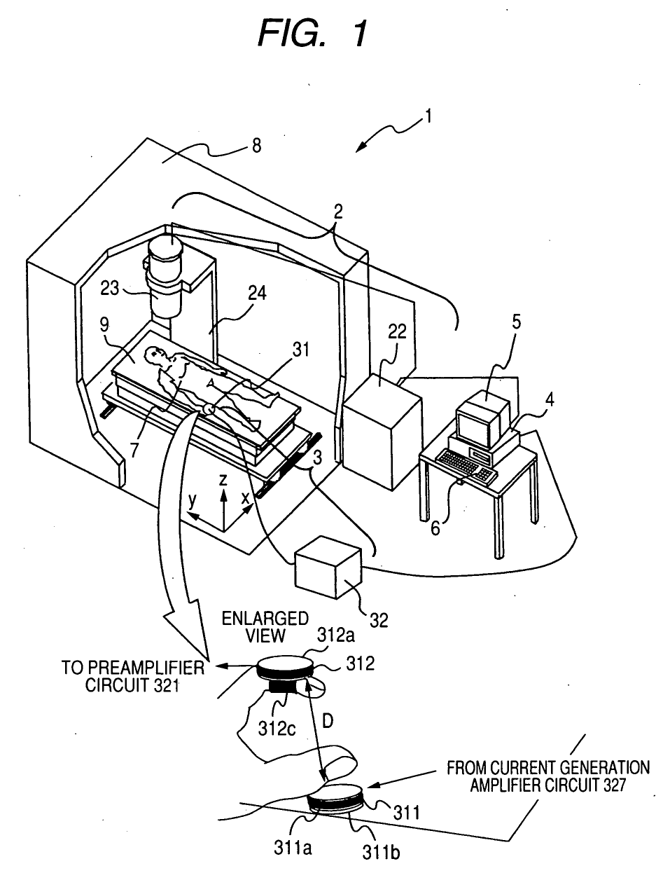

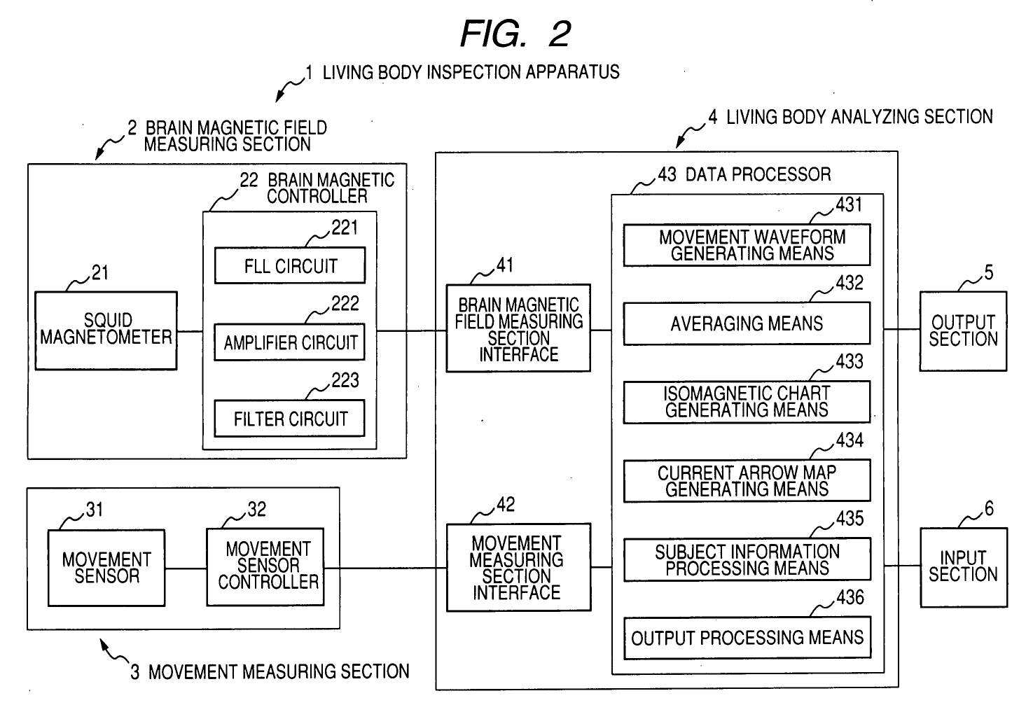

FIG. 1 is a perspective external view of living body inspection apparatus according to the first embodiment; and FIG. 2 is a block diagram showing the general structure of the living body inspection apparatus according to the first embodiment.

As shown in FIGS. 1 and 2, living body inspection apparatus 1 includes: a brain magnetic field measuring section 2 which measures weak magnetic fields emanating from the brain of a subject 7; a movement measuring section 3 which measures finger movements of the subject 7; a living body analyzing section 4 which records and analyzes measurement data obtained by the brain magnetic field measuring section 2 and the movement measuring section 3; an output section 5 which outputs measurement results and analysis results; and an input section 6 which is used to enter information of the subject 7.

Here, the subject 7 is an object which is measured by the brain magnetic field measuring section 2 and the movement measuring section 3. As it makes a movement, electric activity occurs inside it. In other words, in this embodiment, the subject 7 is a living body such as an animal or a human being.

The subject 7 lies down on a bed 9 for supporting the subject 7, installed in a magnetically shielded room 8 which eliminates the influence of environmental magnetic noise. Referring to FIG. 1, the bed 9 is movable so that its position can be adjusted, but during measurement it is fixed on the floor. Here, an orthogonal coordinate system (x, y, z) (magnetic field components are expressed by Bx, By, and Bz) is established so that the xy plane coincides with the plane of the bed 9. The bed 9 need not be horizontal.

In this embodiment, raw data obtained by the brain magnetic field measuring section 2 is called “brain magnetic data” and brain magnetic data averaged by the living body analyzing section 4 is called “brain magnetic waveform.

[Brain Magnetic Field Measuring Section]

The brain magnetic field measuring section 2 acquires brain magnetic data by measuring brain magnetic fields emanating from the subject 7 in time series. For example, the brain magnetic field measuring section 2 may be a biomagnetic field measuring device such as a magnetoencephalograph which is commonly used.

The brain magnetic field measuring section 2 is mainly composed of: a dewar 23 which incorporates a plurality of SQUID magnetometers 21 (FIG. 2) for detecting weak brain magnetic fields; and a brain magnetic controller 22 which controls the SQUID magnetometers 21.

The dewar 23 is intended to cool the SQUID magnetometers 21 to a cryogenic temperature to maintain a superconductive state and inside the dewar 23, each SQUID magnetometer 21 is surrounded by refrigerant such as liquid helium or liquid nitrogen. In order to prevent infiltration of heat from outside, a vacuum layer is formed between the outer wall of the dewar 23 and the refrigerant. The dewar 23 is located above the head of the subject 7 and supported by a gantry 24 standing erect from the floor.

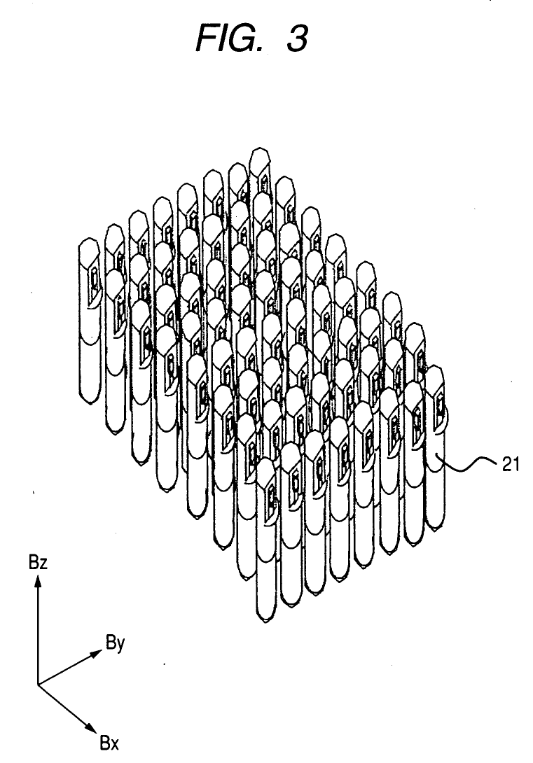

FIG. 3 is a perspective view showing an array of SQUID magnetometers 21. The SQUID magnetometers 2 are sensors which measure the magnetic field component (Bz) perpendicular to a body surface. As shown in FIG. 3, a plurality of SQUID magnetometers 21 stand vertical on the inner wall of the bottom of the dewar 23 (FIG. 1) along the z direction. The plural SQUID magnetometers 21 are equally spaced in the x and y directions so as to detect magnetic change with distance accurately. In this embodiment, for example, the distance between SQUID magnetometers 21 is 25 mm and the number of SQUID magnetometers 21 is 64 (channels) where they are arranged in an 8×8 array pattern.

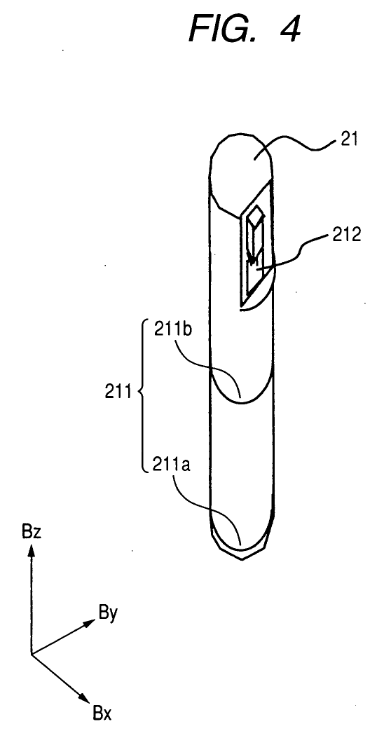

FIG. 4 is a perspective view showing the structure of a SQUID magnetometer 21. As shown in FIG. 4, the SQUID magnetometer 21 includes a primary differential coil 211 and a SQUID 212.

The primary differential coil 211 consists of a detection coil 211a which is located nearer to the subject 7 (FIG. 1), and a reference coil 211b which is located more remotely from the subject 7 than the detection coil 211a and mainly detects external magnetic field noise. The winding direction of the detection coil 211a is opposite to that of the reference coil 211b and the faces of the coils 211a and 21b are oriented in the z direction. In this embodiment, for example, the coil diameter is 20 mm and the distance baseline which represents the interval between coils is 50 mm. External magnetic field noise emanates from a signal source remoter than the subject 7 and this noise is detected by the detection coil 211a and reference coil 211b in the same way. On the other hand, since a signal from the subject 7 is nearer to the coils 211a and 211b than external magnetic field noise, the detection coil 211a detects it as a more intense signal. Therefore, a high S/N ratio is achieved by taking the difference between magnetic forces detected by both the coils 211a and 211b.

The material of the primary differential coil 211 is, for example, superconducting wires such as niobium titanium (Nb—Ti) wires. The primary differential coil (gradiometer coil) 211 transmits the detection result as a magnetic flux to the SQUID 212 so that high sensitive magnetic detection is achieved.

The SQUID 212 is a combination of SQUIDs each made up of a Josephson device coupled with a superconducting ring and a known device may be used for it. The SQUID 212 is connected with the brain magnetic controller 22.

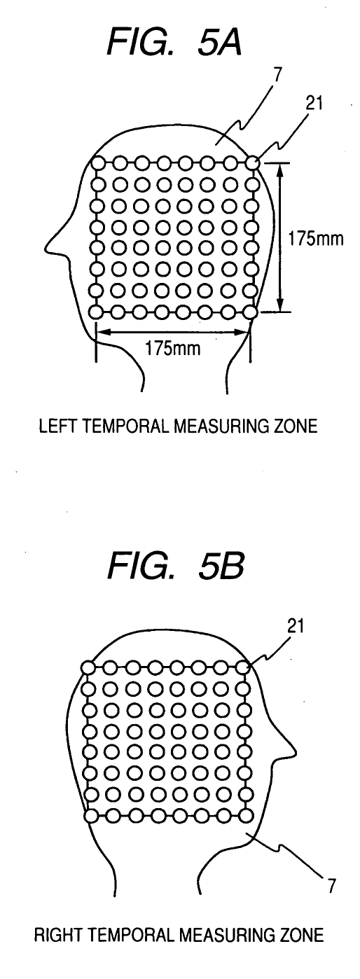

FIGS. 5A and 5B are top views illustrating the positional relation between the head of the subject 7 and the SQUID magnetometers where FIG. 5A shows measurement of the left temporal and FIG. 5B shows measurement of the right temporal. The subject 7 turns his/her head to the right and left respectively. The position of the SQUID magnetometers 21 as shown in FIGS. 5A and 5B is just one example and it can be adjusted as appropriate so that the area to be measured comes in the center of the measuring zone.

As shown in FIGS. 1 and 2, the brain magnetic controller 22 acquires brain magnetic data by controlling the SQUID magnetometers 21. The brain magnetic controller 22 is usually located outside the magnetically shielded room 8 in order to reduce noise detected by the SQUID magnetometers 21.

Next, the process in which the brain magnetic controller 22 acquires brain magnetic data is explained referring to FIG. 2.

As shown in FIG. 2, output from the SQUID magnetometers 21 enters an FLL (Flux Locked Loop) circuit 221 of the brain magnetic controller 22. The FFL circuit 221 lets a current flow through a feedback coil to cancel change in biomagnetic signals entering the SQUID 212 in order to maintain output of the SQUID 212 constant. The current which flows through the feedback coil is converted into a voltage, which is then outputted to an amplifier circuit 222, as a voltage proportional to change in brain magnetic signals.

This output voltage is amplified by the amplifier circuit 222 and filtered by a filter circuit 223 for frequency bandwidth selection, before it is outputted to the living body analyzing section 4.

[Movement Measuring Section]

As shown in FIGS. 1 and 2, the movement measuring section 3 detects movement of the subject 7 in time series and acquires, as waveform data, movement information of the subject 7 related to at least one of the following factors: distance, velocity, acceleration, and jerk. For the movement measuring section 3, a magnetic sensor type tapping device disclosed in JP-A No. 95197/2005 may be used. If a magnetic sensor type tapping device is used, output voltage can be directly used as it is, or without being translated into distance-related data.

The movement measuring section 3 is mainly composed of: a movement sensor 31 consisting of a transmitter coil 311 for transmitting a magnetic field and a receiver coil 312 for receiving the magnetic field; and a movement sensor controller 32.

Referring to FIG. 1, the transmitter coil 311 is put under a thumb, and the receiver coil 312 is put on an upper portion of a forefinger through a band 312c. It is desirable that the band 312c be made of an elastically deformable material such as rubber or sponge so as to absorb finger size differences among individuals.

The transmitter coil 311 is wound on a coil attachment member 311a, and connected to a current generation amplifier 310 in the movement sensor controller 32. The receiver coil 312 is wound on a coil attachment member 312a and connected to a preamplifier circuit 321 in the movement sensor controller 32.

The transmitter coil 311 is fixed on the bed 9 through a fixing means 311b, so that its distance relative to the SQUID magnetometers 21 (i.e. 211a and 211b) is kept constant (at a prescribed value) during measurement. This reduces low frequency noise caused by magnetic field variation which would occur with movement of the transmitter coil 311 when the SQUID magnetometers 21 measure magnetic fields emanating from the patient 7.

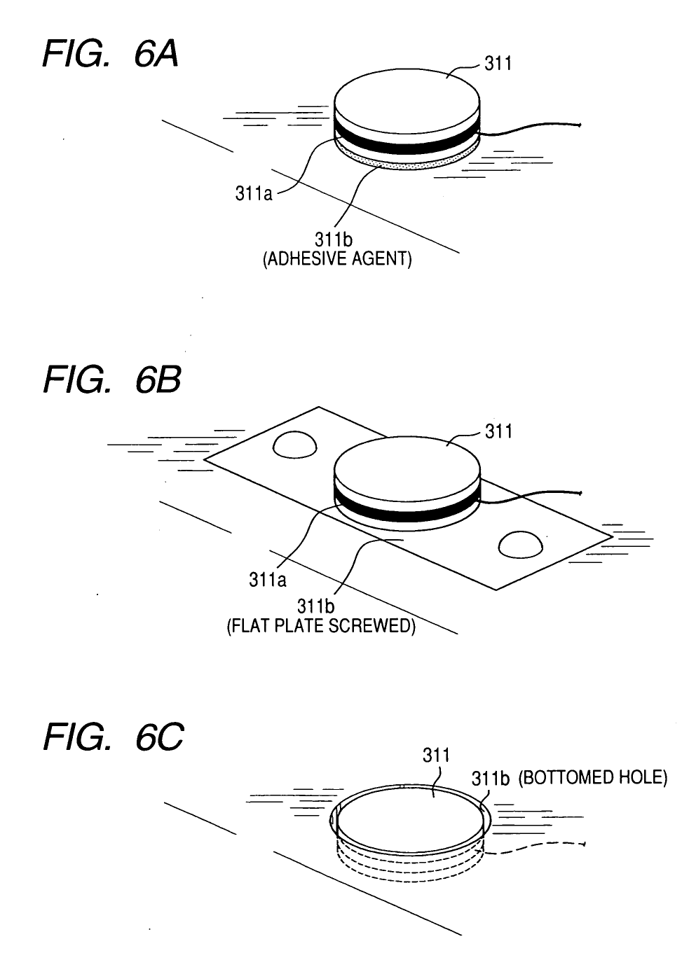

FIGS. 6A, 6B, and 6C are perspective views showing examples of the fixing means 311b.

The structure of the fixing means 311b is not limited as far as the coil attachment member 311a on which the transmitter coil 311 is wound can be fixed on the bed 9 (FIG. 1). One example of the fixing means 311b is as shown in FIG. 6A where it is bonded to the bed with an adhesive agent. Another example is as shown in FIG. 6B where a flat plate protruding from the coil attachment member 311a is screwed on the bed 9. A further example is as shown in FIG. 6C where the transmitter coil 311 is embedded in a bottomed hole made in the bed 9.

The fixing means 311b for fixing the transmitter coil 311 may be such that its position can be adjusted as appropriate to allow the subject to make a movement easily. Even in this case, the positional relationship with the SQUID magnetometers 21 should be constant during measurement.

The finger which is put on the transmitter coil 311 and the finger that wears the receiver coil 312 are not limited to the thumb and the forefinger respectively, but the transmitter coil 311 and the receiver coil 312 may be attached to any fingers. Also, their use is not limited to fingers but they may be attached to four limbs or be designed to measure total body movement. Even in this case, the transmitter coil 311 is fixed on the bed or the like and the transmitter coil 312 is attached to any desired body part of the subject so that the movement of the desired body part is easily measured.

The movement sensor controller 32 (FIG. 2) controls the movement sensor 31 and acquires waveform data relating to finger movement through the movement sensor 31. In order to reduce noise which the SQUID magnetometers 21 detect, it is desirable to install the movement sensor controller 32 outside the magnetically shielded room 8.

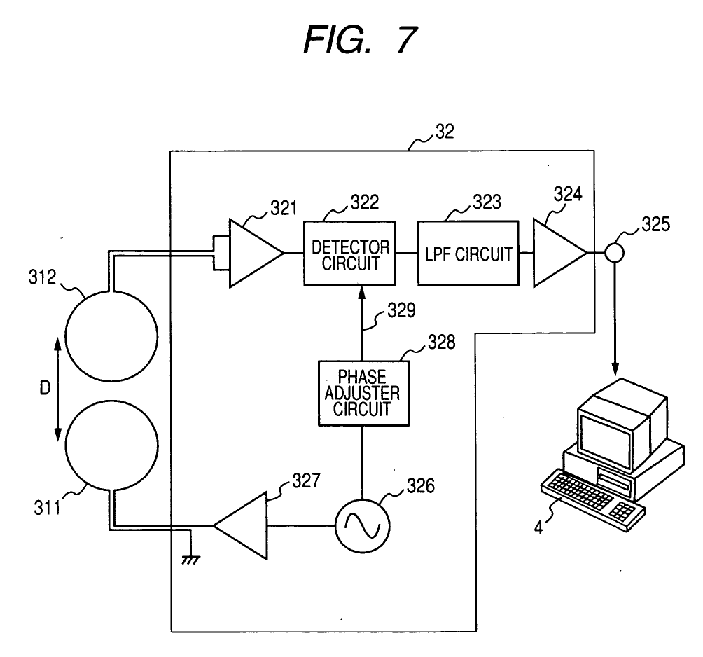

FIG. 7 is a block diagram showing the structure of the movement sensor controller 32. The process in which the movement sensor controller 32 acquires waveform data is explained below referring to FIG. 7.

As shown in FIG. 7, an AC voltage having a specific frequency (for example, 20 kHz, etc.) is developed by an AC generator circuit 326. The AC voltage having the specific frequency which has been developed by the AC generator circuit 326 is converted into an AC current having a specific frequency by a current generation amplifier circuit 327. The AC current that has been developed by the current generation amplifier circuit 327 is allowed to flow in the transmitter coil 311. A magnetic field that has been developed by the transmitter coil 311 develops an induced electromotive force within the receiver coil 312.

The developed induced electromotive force (having the same frequency as that of the AC voltage having the specific frequency which has been developed by the AC generator circuit 326) is amplified by a preamplifier circuit 321, and a signal that has been amplified is sent to a detector circuit 304.

In the detector circuit 322, since detection is made by a specific frequency or double frequency which has been generated by the AC generator circuit 326, an output of the AC generator circuit 326 is connected to a reference signal input terminal of the detector circuit 322 as a reference signal 329 after being adjusted in phase by a phase adjuster circuit 328.

Also, in the case of making detection by double frequency, a frequency which is twice as high as the specific frequency, the phase adjuster circuit 328 is not always required. For a simple circuit structure that makes detection by double frequency, the specific frequency of the AC generator circuit 326 is doubled, and after the frequency is halved by a divider, the frequency is sent to the current generation amplifier circuit 327. The signal having a frequency twice as high as the specific frequency of the AC generator circuit 326 is connected to the reference signal input terminal of the detector circuit 322 as the reference signal 329.

The output of the detector circuit 322 passes through an LPF (low-pass filter) circuit 323, and is then amplified by an amplifier 324 in order to obtain a desired voltage and the amplified output 325 is sent to the living body analyzing section 4. The output 325 is a voltage corresponding to a relative distance D between the receiver coil 312 and the transmitter coil 312 which are put on a living body.

Although the above explanation assumes that a magnetic sensor type tapping device is used as the movement sensor 31, the movement sensor 31 is not limited thereto but may be in any form as far as it is capable of measuring movement by the use of generated magnetic fields. For example, a known, conventional device like a strain gauge or an accelerometer may be used as well. However, in any case, the positional relationship with the SQUID magnetometers 21 should be maintained constant.

[Living Body Analyzing Section]

As shown in FIG. 2, the living body analyzing section 4 records and analyzes measurement data obtained by the brain magnetic field measuring section 2 and the movement measuring section 3.

The living body analyzing section 4 includes a magnetic field measuring section interface 41, a movement measuring section interface 42 and a data processor 43.

The brain magnetic field measuring section interface 41 and the movement measuring section interface 42, which include, for example, an analog digital converter board (hereinafter referred to as “AD board”) as provided in a general computer, convert the brain magnetic data and movement-related waveform data as analog signals detected by the brain magnetic field measuring section 2 and movement measuring section 3, into digital signals at a given sampling frequency, and send them to the data processor 43.

The data processor 43 analyzes the motor function of the subject based on the data acquired by the brain magnetic field measuring section interface 41 and the movement measuring section interface 42 and output the analyzed motor function data to the output section 5 together with the information of the subject.

The data processor 43 includes: movement waveform generating means 431, averaging means 432, isomagnetic chart generating means 433, current arrow map generating means 434, subject information processing means 435, and output processing means 436.

The data processor 43 includes a CPU (Central Processing Unit), a memory that is made up of a ROM (read only memory) or a RAM (random access memory), and a hard disk. The various means 431 to 436 in the data processor 43 are realized when a program or data stored in the memory or the hard disk is loaded in a computer (not shown). The CPU reads the program from the memory and executes arithmetic processing to perform various functions of the data processor 43.

[Movement Waveform Generating Means]

The waveform data that has been acquired from the movement measuring section 3 does not directly express a movement waveform, but expresses a voltage output corresponding to a movement waveform.

The movement waveform generating means 431 converts the waveform data as a voltage output into a corresponding movement waveform, and time-differentiates or time-integrates the converted movement waveform to generate a distance waveform, a velocity waveform, an acceleration waveform, and a jerk waveform in a complementary manner.

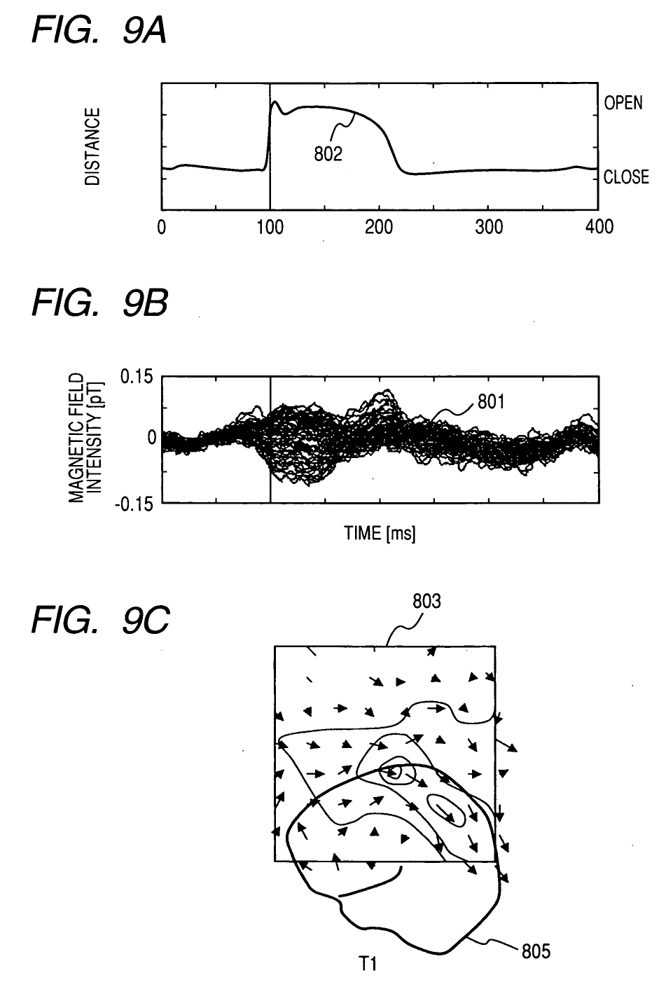

For example, a distance waveform is as indicated by numeral 802 in FIG. 9A.

A “movement waveform” includes at least one of the following types of waveform unless otherwise specified: distance waveform, velocity waveform, acceleration waveform, and jerk waveform.

Even when a strain gauge or velocity meter is used for the movement measuring section 3, if at least one type of movement waveform is measured, other types of movement waveform (distance, velocity, acceleration, jerk) can be obtained by differential and integral calculus in a complementary manner.

[Averaging Means]

The averaging means 432 extracts a region corresponding to a specific movement from brain magnetic data and performs averaging operation on it to generate a brain magnetic waveform.

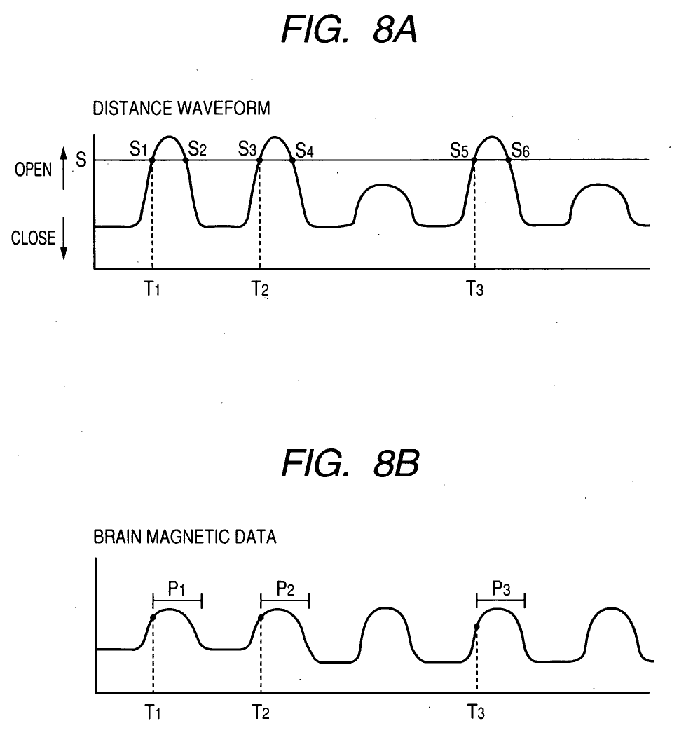

Next, the process in which the averaging means 432 in the first embodiment performs averaging operation on brain magnetic data and generates a brain magnetic waveform is explained referring to FIGS. 8A and 8B.

First, the averaging means 432 extracts intersecting points (S₁ to S₆) at which the movement waveform shown in FIG. 8A intersects with a given threshold S.

Then, the averaging means 432 compares waveform regions before and after the extracted intersecting points (S₁ to S₆) and extracts times (T₁ to T₃) of intersecting points which are in a rising zone (an opening movement is underway). Hereinafter these plural times are treated as additive synchronization points.

The averaging means 432 superimposes these plural additive synchronization points (T₁ to T₃) on the brain magnetic data shown in FIG. 8B and extracts waveform portions (P₁ to P₃) which have a given time width from the additive synchronizing points.

Then, the averaging means 432 performs averaging operation on the extracted plural waveform portions (P₁ to P₃) to obtain a single brain magnetic waveform.

From brain magnetic data obtained by all the SQUID magnetometers 21, brain magnetic waveforms are generated in the same way as mentioned above and a total of sixty-four brain magnetic waveforms are obtained. One example of the result of superimposing the generated sixty-four brain magnetic waveforms is shown in FIG. 9B and this is used to evaluate the magnetic field tendency of a measured body area as a whole.

Thus, the averaging means 432 selects movements with reference to the threshold S and performs averaging operation only on uniform movements, so that movements are adequately related to biomagnetic fields.

[Isomagnetic Chart Generating Means]

The isomagnetic chart generating means 433 extracts magnetic fields at specific times of brain magnetic waveforms and connects equal magnetic fields to draw isomagnetic lines (generally called a magnetoencephalogram, or MEG). These functions performed by the isomagnetic chart generating means are known in conventional brain magnetometers.

[Current Arrow Map Generating Means]

The current arrow map generating means 434 visualizes a pseudo-current by partial differentiation of a magnetic field (Bz) in the z direction perpendicular to the body along the x and y directions.

The concrete partial differentiation method is expressed by the following equations (1) and (2).
Ix=dBz/dy (1)
Iy=−dBz/dx (2)

Here, a current arrow which represents a pseudo current is shown as an arrow (Ix, Iy) on the xy plane.

FIG. 9C is a current arrow map showing brain magnetic waveforms according to this embodiment. In FIG. 9C, reference numeral 803 represents a measuring screen and reference numeral 805 represents the brain of the subject 7. As shown in FIG. 9C, the current arrow map at time T1 suggests that intensive currents have been detected in the somatosensory area in the vicinity of the top of the head. On the other hand, no currents have been detected in the auditory sensory area as shown at the bottom of the diagnosis screen 803.

Thus, the current arrow map generating means 434 quantifies an electrophysiologic excitation transmission process without dipole estimation or displaying many isomagnetic charts.

Although the current arrow map method has been described as a method of making a current distribution diagram in this embodiment, the invention is not limited to the current arrow map method. For instance, a diagram similar to that in FIG. 9C can be drawn up using the minimum norm method or lead field method.

[Subject Information Processing Section]

The subject information processing section 435 (FIG. 2) is provided with a subject database (not shown) which records such information as subject information and analysis results and manages information recorded in the subject database.

More specifically, in the case of conducting 1) registration, correction, deletion, retrieval, and sorting of the subject information, 2) association of the subject information with the measurement data, 3) registration, correction, and deletion of the analysis result of the measurement data (addition, correction, and deletion of items), and 4) statistical processing, the subject information processing section 435 processes main four items related to the registration, correction, and deletion of the statistical processing results in conjunction with the subject database.

Also, the subject information to be recorded in the subject database includes subject ID, name, birth date, age, body height, body weight, disorder name, and comments on the subject.

The information management as mentioned above can be readily made by the subject information processing section 435 using the conventional program and data configuration.

Also, a hard disk or the like may be used for the subject database.

[Output Processing Means]

The output processing means 436 displays information recorded in the subject database such as subject information or analysis results on the output section 5 in a visually understandable manner, in the form of graphs or tables as appropriate.

The output processing section 436 does not have to display all the above analysis results at the same time, and may display items that are selected by the operator as appropriate.

The output section 5 outputs subject information and movement information which are processed by the data processor 43 and may be embodied, for example, as an LCD (Liquid Crystal Display), CRT (Cathode Ray Tube) display, or printer.

The input section 6 is intended to enable the operator (not shown) of the living body inspection apparatus 1 to enter subject information, etc. and may be embodied as a keyboard, mouse or the like. When the operator enters subject information etc., an input screen may appear on the display as a user interface which helps the operator make an entry.

### Second Embodiment

Next, living body inspection apparatus according to the second embodiment will be described referring to relevant drawings. In the second embodiment, the motor function of the subject is measured while the subject is tapping in response to sound stimulation. Concretely, the subject is instructed to make tapping movement by putting together his/her forefinger and thumb in tune with sound issued from a sound stimulation device.

In the description of the second embodiment given below, features characteristic thereof are described in detail but description of other features which are the same as in the first embodiment is omitted.

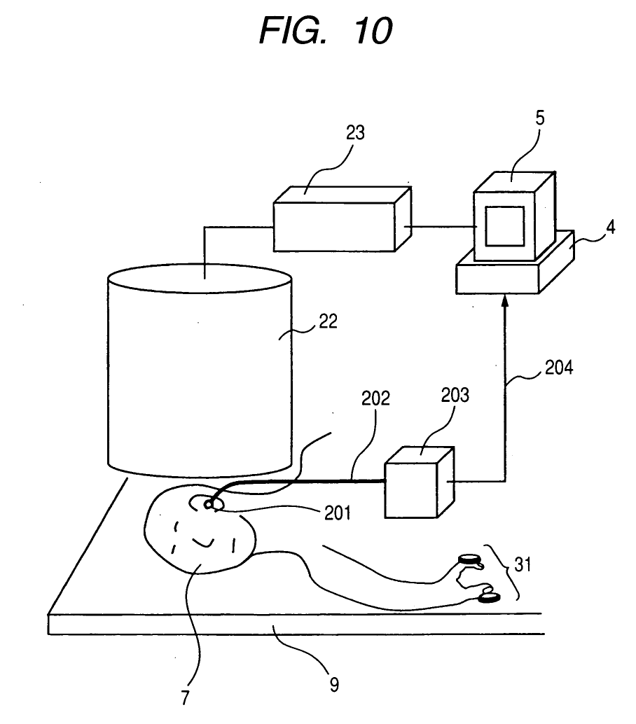

As shown in FIG. 10, living body inspection apparatus 1 includes: a brain magnetic field measuring section 2 (FIG. 1) which measures weak magnetic fields emanating from the brain of the subject 7; a movement measuring section 3 (FIG. 1) which measures finger movements of the subject 7; a living body analyzing section 4 which records and analyzes measurement data obtained by the brain magnetic field measuring section 2 and the movement measuring section 3; an output section 5 which outputs measurement results and analysis results; an input section 6 which is used to enter information of the subject 7; and a sound stimulation device 203 which gives a sound stimulus to the subject 7.

The sound stimulation device 203 generates tone burst sound and sends it to the subject 7 and also generates a synchronizing signal 204 and sends it to the living body analyzing section 4.

For example, the tone burst sound generated by the sound stimulation device 203 is retained for 50 ms at 1 kHz and a sound stimulus is given at intervals of 0.3 Hz (or every 3.3 seconds). The tone burst sound generated by the sound stimulation device 203 is sent through an air tube 202 and an adapter 201 to an ear of the subject.

Though not shown in FIG. 10, white noise is constantly given to the right ear during measurement in order to prevent any influence of an external sound.

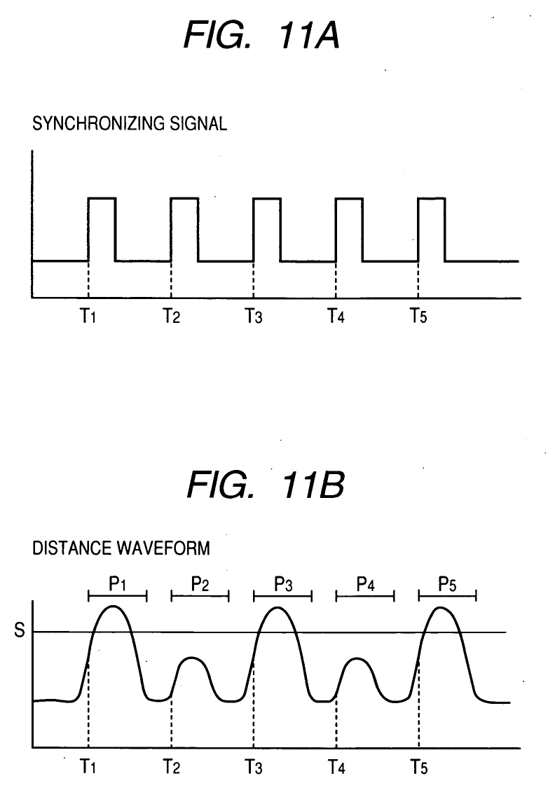

Next, the process in which the averaging means 432 in the second embodiment performs averaging operation on brain magnetic data to generate a brain magnetic waveform will be explained, referring to FIGS. 11A and 11B.

The averaging means 432 of the data processor 43 in the second embodiment uses synchronizing signal times (T₁ to T₅) as shown in FIG. 11A as additive synchronizing points in performing averaging operation on brain magnetic data. Even in this case, a given threshold S is set for a distance waveform shown in FIG. 11B, so that among the waveform portions (P₁ to P₅) which have a given time width from the additive synchronizing points (T₁ to T₅), averaging operation is performed only on brain magnetic data (not shown) corresponding to the waveform portions beyond the threshold S, namely (P₁, P₃, P₅).

When the motor function is measured by the use of sound stimulation in this way, the motor function can be evaluated in a condition that the auditory sensory area and the somatosensory area are activated simultaneously.

Also, the use of the sound stimulation device 203 makes it possible that averaging operation is done on brain magnetic data according to the synchronizing signal 204 generated by the sound stimulation device 203.

### Other Embodiments

The form in which the present invention is embodied is not limited to the first and second embodiments as mentioned above and it may be embodied in other various forms within the scope of its technical idea.

In the above embodiments, the transmitter coil 311 of the movement sensor 31 is fixed on the bed 9; however, it need not be always fixed on the bed 9. For example, it may be fixed on the dewar 23, gantry 24 or a fixture (not shown) protruding directly from the floor.

In the above embodiments, an isomagnetic chart and an arrow map are generated based on averaged brain magnetic waveforms to evaluate the motor function of the subject 7; however, the invention is not limited thereto. For example, the motor function of the subject 7 may be evaluated by dipole assumption which uses a conventional known algorithm.

Instead of being outputted directly as it is, the analysis result from the data processor may be statistically processed before being outputted. In this case, a statistical processing section may be included in the data processor and the analysis result is divided into groups (for example, a healthy subject group and different disease groups) and statistically processed (for example, for calculation of average or variance values).

In the above embodiments, the brain magnetic field of a subject in motion is measured using a brain magnetometer as a biomagnetic field measuring device; however, any other device that is capable of measuring biomagnetic fields may be used. For example, a heart magnetometer, muscle magnetometer or lung magnetometer may be used instead of a brain magnetometer.

In the above embodiments, the S/N ratio is improved by performing averaging operation on brain magnetic data; however, it is obvious that a known method may be combined to further improve the S/N ratio.

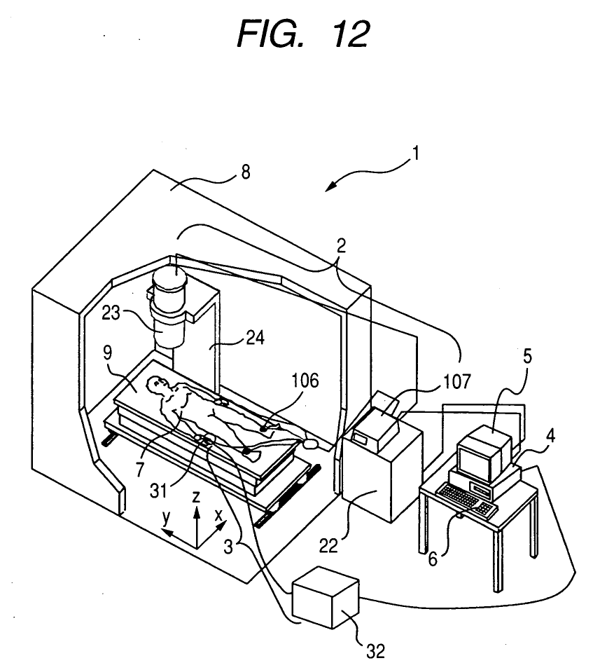

FIG. 12 illustrates a case that an electrocardiograph is used along with the living body inspection apparatus 1. As shown in FIG. 12, electrodes 106 for the electrocardiograph are attached to the subject's four limbs and these electrodes 106 are connected with the electrocardiograph 107. The electrocardiograph 107 can use the amplifier circuit 222 and filter circuit 223 (FIG. 2) of the brain magnetic controller 22 as appropriate.

This compensates for an influence of a magnetic field signal from the heart included in the brain magnetic data. By performing averaging operation on the compensated brain magnetic data, a more appropriate brain magnetic waveform can be generated.

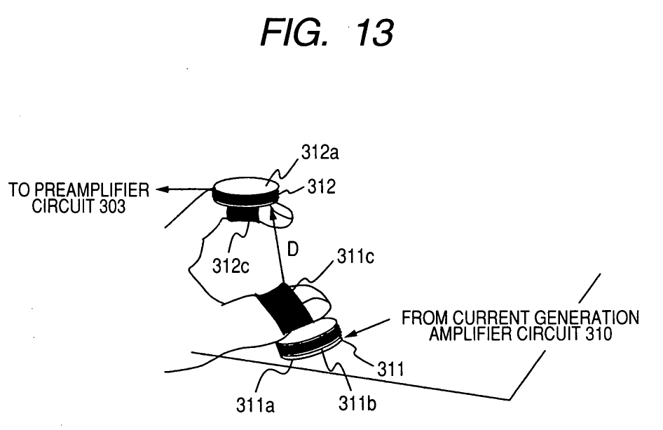

In the above embodiments, the transmitter coil 311 is fixed on the bed 9; however, it may be fixed on the thumb through coil attachment means 311c as shown in FIG. 13.

What is claimed is:

1. Living body inspection apparatus comprising:
a movement sensor which includes magnetic field generating means for generating a magnetic field and magnetic field receiving means attached to a living body for receiving the magnetic field and measures movement of the living body based on the magnetic field emitted from the magnetic field generating means and received by the magnetic field receiving means;
a SQUID magnetometer which receives a biomagnetic field emanating from the living body;
analyzing means which analyzes biomagnetic field measurement data obtained by the movement sensor and the SQUID magnetometer; and
display means which outputs a result of analysis made by the analyzing means.

2. The living body inspection apparatus as described in claim 1, wherein the magnetic field generating means is fixed in a prescribed position relative to the SQUID magnetometer.

3. The living body inspection apparatus as described in claim 1, wherein the magnetic field generating means is fixed on a support platform for supporting the living body.

4. The living body inspection apparatus as described in claim 1, wherein the analyzing means includes:
movement waveform generating means which generates, from time series waveform data acquired by the movement sensor, a movement waveform corresponding to the waveform data; and
averaging means which sorts the biomagnetic field data based on the movement waveform and performs averaging operation on selected biomagnetic field data to generate a biomagnetic filed waveform.

5. The living body inspection apparatus as described in claim 4, wherein the analyzing means further includes isomagnetic chart generating means for generating an isomagnetic chart based on the biomagnetic field waveform.

6. The living body inspection apparatus as described in claim 4, wherein the analyzing means further includes current distribution chart generating means for generating a current distribution chart based on the biomagnetic field waveform.
